(19) United States
(12) Plant Patent Application Publication
(10) Pub. No.: US 2013/0326775 P1
(43) Pub. Date: Dec. 5, 2013

(54) Clematis Plant Named 'Evipo055'

(50) ;

(71) Applicants: Mogens Nyegaard Olesen, Fredensborg (DK); Raymond J. Evison, St. Sampsons (GB)

(72) Inventors: Mogens Nyegaard Olesen, Fredensborg (DK); Raymond J. Evison, St. Sampsons (GB)

(21) Appl. No.: 13/507,014

(22) Filed: May 31, 2012

(51) Int. Cl. A01H 5/00

(52) U.S. Cl. PLT/228

(57) ABSTRACT

A new Clematis plant with a compact growth habit, profuse, white flowers, and continuous summer flowering. The variety successfully propagates from softwood cuttings and is suitable for cultivation commercial nursery culture. This new and distinct variety has shown to be uniform and stable in the resulting generations from asexual propagation from vegetative cuttings.

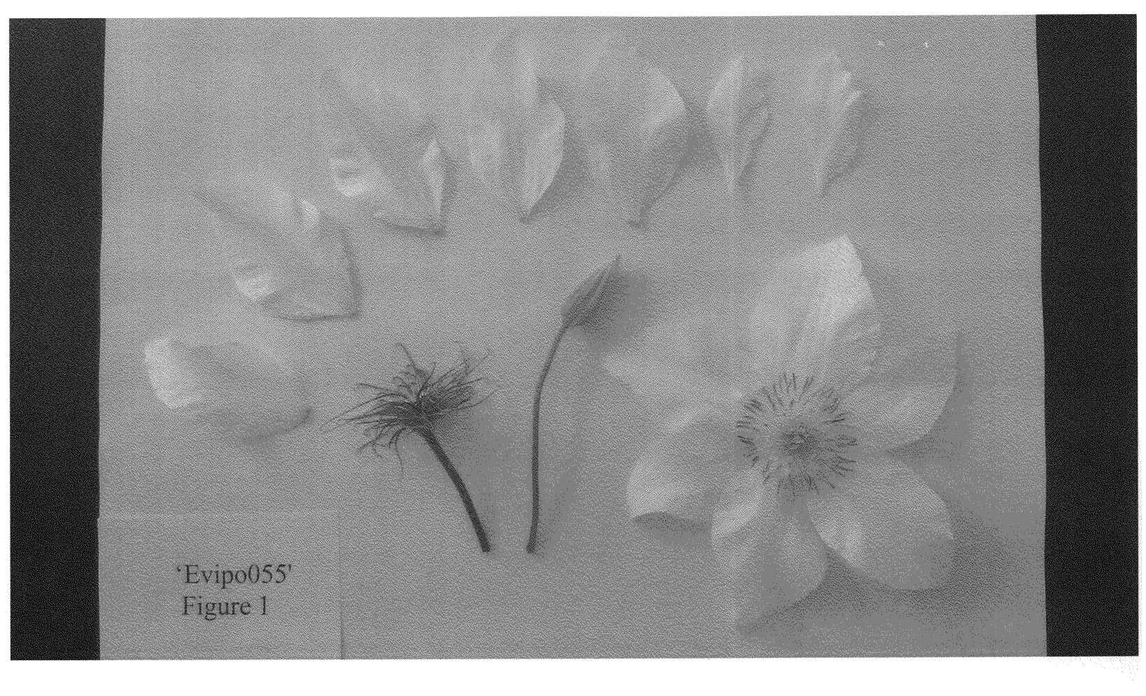

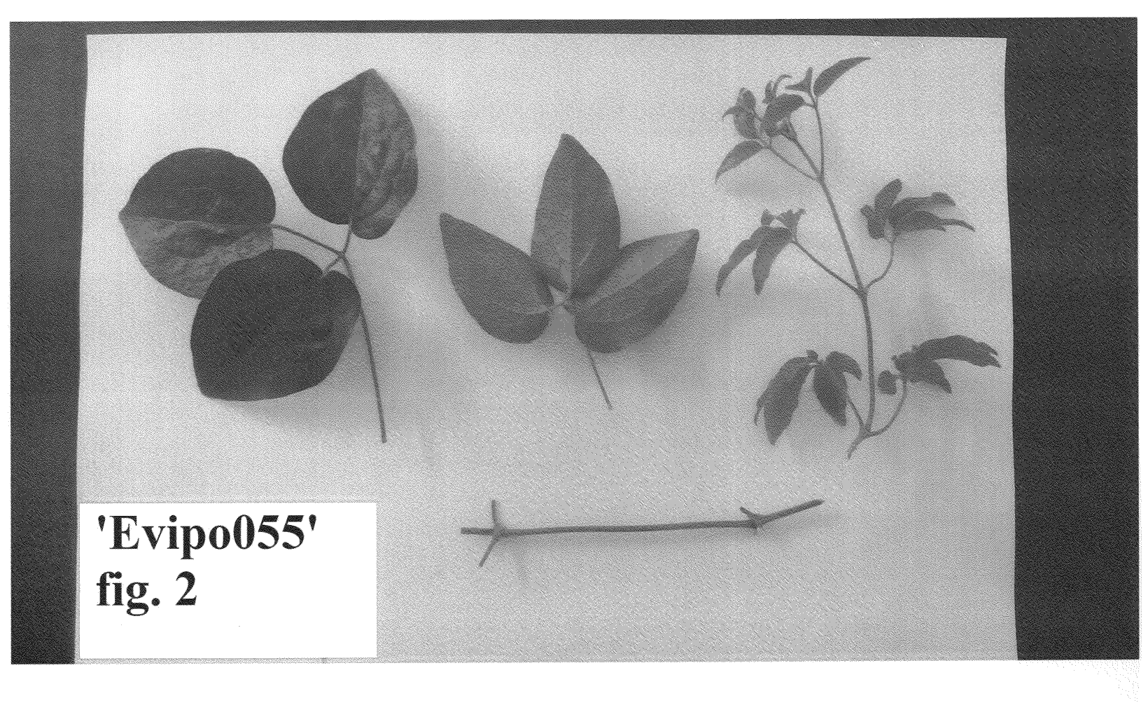

### BOTANICAL CLASSIFICATION

Genus.—Clematis

### SPECIES

viticella

### VARIETY DENOMINATION

‘Evipo055’

### SUMMARY OF THE CLAIMED PLANT

The present invention constitutes a new and distinct variety of Clematis plant which originated from a controlled crossing between the female seed parent, an unnamed seedling, and the male pollen parent, an un-named seedling

The two parents were crossed during the summer of 1994 and the resulting seeds were planted the following winter in a controlled environment in Guernsey, Channel Islands. The new variety named ‘Evipo055’ originated as a single seedling from the stated cross.

The new clematis plant may be distinguished from its female seed parent and male pollen parent primarily by flower color and growth habit.

The objective of the hybridization of this clematis plant was to create a new and distinct variety for nursery culture with unique qualities such as:

1. Uniform and abundant white flowers;

2. Vigorous and compact growth, making the variety suitable for container culture; and

3. Improved disease resistance.

This combination of qualities was lacking in clematis plants that were in commercial cultivation and the qualities have been substantially achieved in the new variety.

‘Evipo055’ was selected by Mogens N. Olesen and Raymond J. Evison in their clematis development program in the Channel Islands, United Kingdom in [selection year]. Asexual reproduction of ‘Evipo055’ by means of vegetative cuttings and traditional layering was first performed by Mogens N. Olesen and Raymond J. Evison in the nursery during the summer of 1995. This initial and subsequent asexual propagations have demonstrated that the characteristics of ‘Evipo055’ are true to type and are transmitted from one generation to the next.

### BRIEF DESCRIPTION OF THE DRAWING

The accompanying color illustration show as true as is reasonably possible to obtain in color photographs of this type the typical characteristics of the buds, flowers, leaves, and stems, of ‘Evipo055’. Specifically illustrated in the drawing are flowers at various stages of development, flower in parts, leaves, and stems.

### DETAILED DESCRIPTION OF THE VARIETY

The following is a detailed description of ‘Evipo055’, as observed in its growth throughout the flowering period in glasshouses the Netherlands. Observed plants were cultivated for a period of 18 months in 17 cm containers. Certain phenotypical characteristics of the variety may vary under different environmental, cultural, agronomic, seasonal, and climatic conditions. Color references are made using The Royal Horticultural Society (London, England) Colour Chart, 2001, except where common terms of color are used.

For a comparison, several physical characteristics of the clematis variety ‘Evitwo’ described and illustrated in U.S. Plant Pat. No. 10,167 are compared to ‘Evipo055’ in Chart 1.

CHART 1

| | ‘Evipo055’ | ‘Evitwo’ |
|---|---|---|
| Flower diameter | 135 mm. | 100 to 180 mm |
| General tonality | White Group 155D | White Group 155C |
| of flower color | | |
| Tepal count | 6 | 12 |

Flower and flower bud:
   Blooming habit.—Continuous. The natural flowering period is generally from April to September. Flowers on new and old growth.
   Flower bud.—Size: Length 28 mm, diameter 14 mm. Tepal color: At ¼ opening, White Group 155B.
   Bud form.—Elliptic.
   Peduncle.—Length: 75 mm. Diameter: About 1 mm.
   Flower arrangement.—Borne: Singly.
   Flower bloom.—Size: Diameter 135 mm. 10 mm deep. Shape from the side: Flat. Fragrance: None. Lasting Quality: Flowers normally remain 10 days on the plant. General Tonality: White Group 155D.
   Tepals.—Upon opening: Upper surface is White Group155D. Lower surface is White Group 155C. After opening: Upper surface is White Group155D. Lower surface is White Group 155C. Quantity: Normally 6 tepals. Size: 65 mm in length by 37 mm wide.
   Shape.—Individual tepal shape is elliptic. The tepal apex is acuminate. The tepal base is typically acute. Apex Recurvature: Slightly curved/recurved. Margins: Entire. Weak undulations of margin observed. Persistence: Tepals drop off cleanly.
Plant:
   Size.—Seasons growth attains 1.8 meters in height. About 45 cm wide.
   Stems.—Color: Juvenile stems are Yellow-Green Group 145B. Mature stems are Greyed-Orange Group 166B. Internodes: On average, 7 cm between nodes. Length: Normally 50 to 80 cm from the base of the plant to the flowering portion of the stem. Diameter: About 3 mm. Texture: Mature stems are ribbed. Juvenile stems are smooth.
   Plant foliage.—Arrangement: Ternate. Leaf characteristics: Deciduous. Leaf Size: Compound leaves are 80 mm (l)×120 mm (w). Leaflets are normally 55 mm (l)×45 mm (w). Abundance: On average 2 leaves per 10 cm of stem.
   Leaf color.—Juvenile upper Yellow-Green Group 144A. Juvenile lower Yellow-Green Group 144A. Mature upper Yellow-Green Group 146A. Mature lower Yellow-Green Group 146B.
   Leaflet shape.—Generally cordate. The base is cordate to rounded, the apex is acute to widely acute. Margin: Entire. Surface: The upper side is smooth. The lower side is smooth. Thickness: Moderate. Glossiness: Moderately Glossy.
   Disease resistance.—Subject to any disease that normally attacks the species. However the variety is more tolerant to clematis wilt than some clematis.
   Cold hardiness.—The variety is tolerant to USDA Hardiness Zone 6.
   Heat tolerance.—The variety has been found to be suitable for climate conditions found in the American Horticulture Society heat zone 7.

1. We claim a new and distinct variety of clematis plant named ‘Evipo055’, substantially as described and illustrated herein, due to its abundant white flowers with good keepability, attractive foliage and compact growth, year round flowering under glasshouse conditions, suitability for production from softwood cuttings in pots, durable flowers and foliage which make the variety suitable for distribution in the floral industry.
